(19) United States
(12) Patent Application Publication
(10) Pub. No.: US 2005/0240219 A1
(43) Pub. Date: Oct. 27, 2005

(54) Peripheral vascular occlusion devices

(76) Inventors: Henry Kahle, Trabuco, CA (US); Ghassan Sakakine, Mission Viejo, CA (US)

Correspondence Address: APPLIED MEDICAL RESOUCES CORPORATION, 22872 Avenida Empresa, Rancho Santa Margarita, CA, 92688, US

(21) Appl. No.: 10/831,049

(22) Filed: Apr. 22, 2004

(51) Int. Cl. A61B 17/28

(52) U.S. Cl. 606/207

(57) ABSTRACT

The invention relates to a surgical instrument for at least partially occluding a body conduit comprising a first jaw, a second jaw opposing the first jaw and moveable relative to the first jaw to occlude the conduit, and an insert being mounted by at least one of the first and second jaws with a length of the insert extending along at least one of the first and second jaws. One of the first and second jaws may further comprise a retention barb to provide a friction fit with the insert. The insert is flexible, disposable and may be formed by injection molding. The retention barb may be a three-sided or multiple-sided barb that is formed at the proximal end of at least one of the first and second jaws. The first and/or second jaws may be bent and/or curved. In another aspect, the insert comprises an outer wall and an inner wall for fitting over the jaw, and the inner wall further comprises a molded, rectangular raised ring formed at the proximal end to aid the retention of the insert over the retention barb of the jaw. The insert may be removed from the jaw by pulling the insert in a distal direction to overcome the friction fit of the retention barb. The insert may further include a soft tip covering the distal end of one of the jaws to minimize trauma during surgery. The distal ends of the jaw and the insert may be tapered to allow ease of installation and removal. In another aspect, the insert and jaws may be removably attached to one another in a snap-fit relationship where the jaws may include one or more holes and the insert may include one or more projections. It is appreciated that the holes and projections may vary in width, depth and height depending on application.

### BACKGROUND OF THE INVENTION

1. Field of the Invention

This invention generally relates to surgical conduit occlusion devices such as clips and clamps and, in particular, to a low profile, atraumatic peripheral occlusion device having a clamp tip and an insert.

2. Discussion of the Prior Art

The human body contains many body conduits that accommodate a flow of fluids and semi-solids among various locations of the body. Typical of such conduits are the intestines and blood vessels including both veins and arteries.

Many surgical procedures require the reduction of flow and, more typically, the occlusion of flow in such body conduits. This occlusion is typically accomplished with devices commonly referred to as clamps or clips. In general, these devices have opposing jaws which are adapted to extend exteriorally over the body conduit and to pinch the walls of the conduit against themselves in order to inhibit or stop flow through the conduit.

The jaws commonly associated with clamps are relatively long and are operable by scissor-type handles that provide leverage for the jaws. Clamps are commonly used to occlude larger conduits such as the intestines. By comparison, the jaws of clips are relatively small and are biased by a spring contained in a telescoping housing. The smaller clips are commonly used to occlude smaller conduits such as vessels. As used herein, the word “clamp” will refer not only to clips and clamps but other types of occlusion devices which have opposing jaws.

Body conduits typically have relatively smooth outer surfaces which are often wetted by body fluids such as blood. Attempts to occlude such conduits by exteriorally pinching their walls must meet two objectives. The first objective is to accomplish the occlusion, and the second objective is to maintain the clamp in place. Herein lies the problem commonly referred to as traction.

Traction in this case relates to the resistance of the clamp to movement relative to the tissue. Theoretically, this traction is equivalent to the product of a coefficient of friction, which is dependent upon the nature of the contacting surfaces, and the normal or clamping force. In the past, attempts have been made to increase this clamping force in order to increase the traction. Unfortunately, squeezing the body conduit with a greater force tends to traumatize the tissue and in the case of vessels, destroy the precious intimal lining which cannot be regenerated. As a result, it is desirable to have only sufficient clamping force to achieve the first objective, that is, the occlusion of the conduit. Increasing the clamping force to achieve the second objective, that is, increase traction, is generally not a suitable alternative.

As an example of the above, many current pediatric vascular clamps utilize metal jaws without inserts to achieve clamping and mobilization of small organs, vessels and tissue. The metal jaws provide a high degree of traction, however, these metal jaws traumatize the immediate and surrounding tissue in the process. It is also possible for the metal jaws to perforate delicate tissue if an excessive clamping force is applied. For at least the above reasons, there is a need in the art for a low profile, atraumatic occlusion device for clamping small organs, vessels and tissue that provides the same high degree of traction as a metal clamp.

### SUMMARY OF THE INVENTION

The invention relates to low profile, atraumatic clamp and insert system to assist in mobilizing and holding small organs, vessels and tissue in a secure yet atraumatic manner during a vascular procedure.

In one aspect of the invention, a surgical instrument for at least partially occluding a body conduit includes a first jaw, a second jaw opposing the first jaw and moveable relative to the first jaw to occlude the conduit, and an insert being mounted by at least one of the first and second jaws with a length of the insert extending along at least one of the first and second jaws. A feature of the invention is at least one of the first and second jaws may further comprise a retention barb to provide a friction fit with the insert. The insert is flexible, disposable and may be formed by injection molding. The retention barb may be a three-sided or a multiple-sided barb that is formed at the proximal end of at least one of the first and second jaws. The first and/or second jaws may be bent or curved.

In another aspect of the invention, the insert comprises an outer wall and an inner wall for fitting over the jaw, and the inner wall further comprises a molded raised ring to aid the retention of the insert over the retention barb of the jaw. With this aspect, the molded raised ring is formed in the shape of a rectangle at the proximal end of the insert. The insert with the molded raised ring may be removed by pulling the insert in a distal direction to overcome the friction fit of the retention barb. In another aspect, the insert may be formed to have a thicker wall and a traction pattern on a clamping surface. The thicker wall operates to prevent rollover and the traction pattern provides atraumatic clamping. In another aspect, the insert further includes a soft tip covering the distal end of one of the jaws to minimize trauma during surgery. In yet another aspect, the distal ends of the jaw and the insert are tapered to allow ease of installation and removal.

In another aspect of the invention, a surgical clamp for at least partially occluding a body conduit is disclosed comprising a first jaw having a top surface and a bottom surface, a second jaw opposing the first jaw having a top surface and a bottom surface and moveable relative to the first jaw to occlude the body conduit, and at least one insert having a top surface and a bottom surface, the insert being attached to the first jaw with the bottom surface of the insert being removably attached to the top surface of the first jaw, wherein the bottom surface of the insert comprises at least one or more projections and the top surface of the first jaw comprises at least one or more holes sized and configured to receive the one or more projections of the insert. In this aspect of the invention, the one or more projections of the insert and the one or more holes of the jaw provide a snap-fit relationship facilitating removable attachment of the insert to the jaw. A feature of this aspect of the invention is each of the one or more projections of the insert may vary in width and height.

These and other features and advantages of the present invention will be more apparent with a description of preferred embodiments in reference to the associated drawings.

### DESCRIPTION OF THE DRAWINGS

FIG. 1 is a perspective view of a surgical clamp of the present invention engaging a body conduit;

FIG. 2 is a side-elevation view of a clamp jaw and system for attaching a disposable insert of the present invention;

FIG. 3 is a side-elevation view of a clamp jaw and system for attachment of a further embodiment of a disposable insert.

FIG. 4 is a side-elevation view illustrating attachment of the insert illustrated in FIG. 3;

FIGS. 5(a)-5(d) illustrate perspective views of a clamp jaw and system for attaching a disposable insert having a plurality of projections in accordance with another embodiment of the invention;

FIG. 6 is a side-elevation view of a further embodiment of an insert illustrating a method of attachment of the invention;

FIGS. 7(a) and 7(b) illustrate perspective views of a clamp jaw having a three-sided barb to aid retention of an insert in accordance with another embodiment of the invention;

FIG. 8 illustrates a cross-section view of an insert having a rectangular molded raised ring for use with the clamp jaw of FIG. 7(a) of the invention; and

FIGS. 9(a) and 9(b) illustrate perspective views of a tapered clamp jaw and insert to allow for ease of installation and removal in accordance with another embodiment of the invention.

### DESCRIPTION OF PREFERRED EMBODIMENTS AND BEST MODE OF THE INVENTION

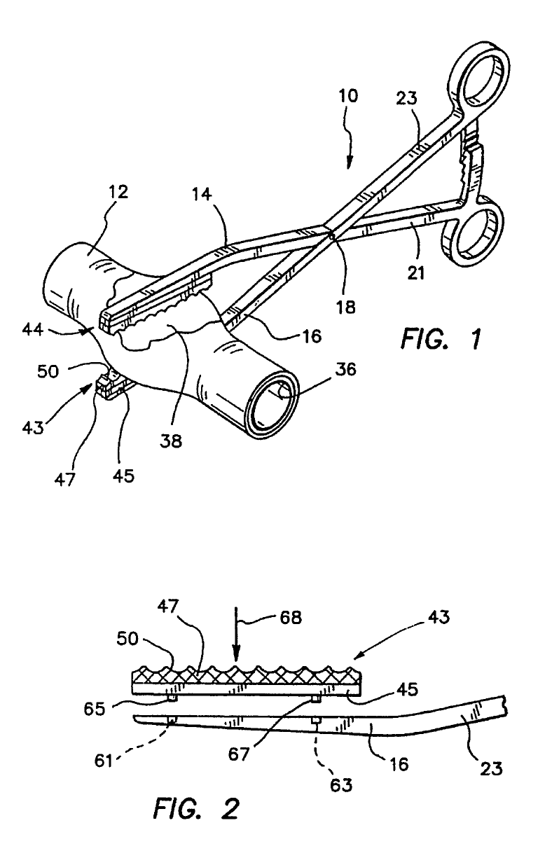

Clamps are commonly used to restrict the flow of fluids and semi-solids in body conduits. By way of example, a clamp 10 is illustrated in FIG. 1 and designated generally by the reference numeral 10. This clamp is illustrated in an operative state occluding a body conduit such as a portion of an intestine 12. The clamp 10 has a pair of opposing jaws 14 and 16 which are pivotal relative to each other at a fulcrum 18 by operation of associated scissor-type handles 21 and 23, respectively. Since the intestine 12 is a relatively large conduit, the clamp 10 will typically have jaws 14, 16 with a length of about two to three inches.

The jaws 14, 16 in the open state are widely separated to facilitate insertion of the associated conduit 12 between the jaws. In the closed state, the jaws 14, 16 are closely spaced to engage the exterior of the conduit 12, and to pinch opposing walls of the conduit 12 against themselves so as to create a restriction in the flow of fluids through the conduit 12. This restriction may result in total occlusion of the conduit 12 if the opposing walls are pinched into contact by the jaws 14, 16 of the clamp 10. Where complete occlusion is desired, substantial clamping forces may be required in order to bring the opposing walls of the conduit 12 into contact. These forces, of course, tend to traumatize the conduit 12 and, importantly in the case of vessels, may damage the inner, intimal lining 36 of the vessel. Since this intimal lining 36 cannot be regenerated, substantial occlusion forces are to be avoided.

While it can be appreciated that reduced clamping forces may be desirable to inhibit trauma to the conduit 12, increased clamping forces may be desirable to facilitate traction with the conduit 12 in order to maintain the clamp 10 in its occlusive state. Traction can be a difficult problem particularly in a surgical environment wherein the outer surface of the conduit 12 will typically be wetted or coated with a liquid such as blood 38.

It is for these reasons that the clamp 10 is designed to address competing requirements for a clamping force reduced to avoid trauma to the conduit 12, and a sheer force increased to facilitate traction between the clamp 10 and the conduit 12.

In the past, trauma has been addressed by providing resilient pads or inserts 44 and 43, one for each of the associated jaws 14 and 16. Each of inserts 44, 43 has commonly included rubber or foam which has provided a relatively soft tissue-contacting surface for jaws 14,16, respectively. In some cases, this tissue-contacting surface has been molded with an irregular shape-facilitating traction surface with the conduit 12. The insert 43 has a generally rigid, such as plastic, substrate or base 45 which is coupled to the associated jaw 16. A resilient pad 47 is attached to the base 45 and overlaid with a cover 50. It is this cover 50, with or without the resilient pad 47, which provides increased traction without an increase in clamping force or a resulting increase in trauma to the conduit 12.

The clamp 10 will typically include reusable jaws 14, 16 in combination with disposable inserts 44, 43. In an embodiment including disposable inserts, attachment of the jaws 16 to the insert 43 can be accomplished with a variety of systems. Typically, the jaw 16 will include a top surface facing toward to the opposing jaw 14, an opposing bottom surface, and a pair of side-surfaces extending therebetween.

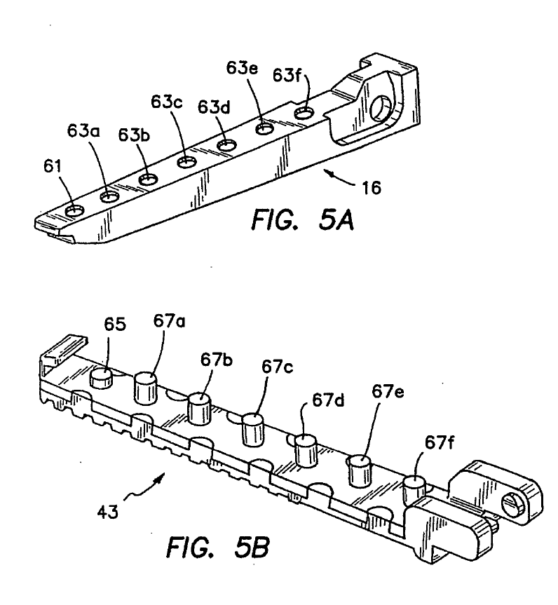

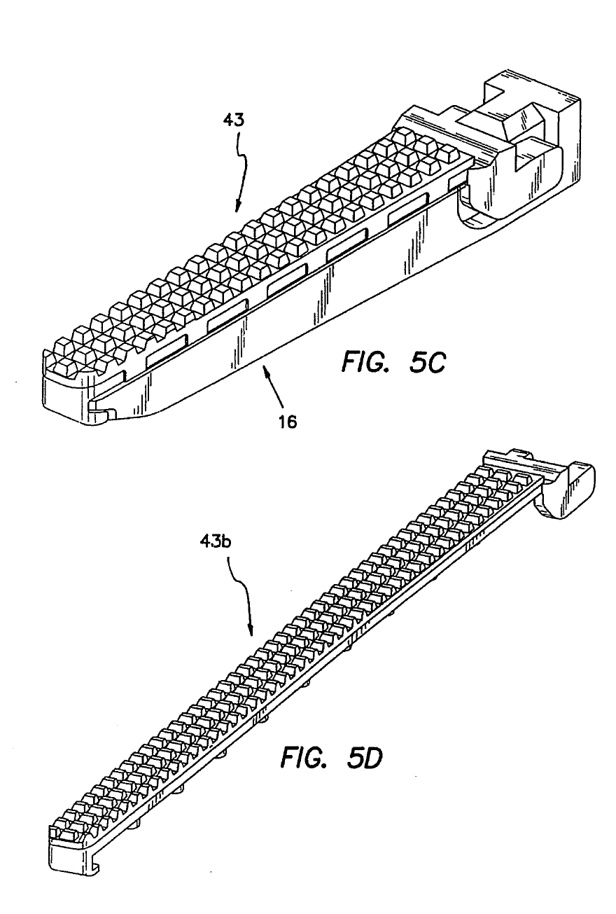

Referring to FIG. 2, there is shown a side-elevation view of a clamp jaw and system for attaching a disposable insert of the present invention. In particular, the top surface is provided with two or more holes 61, 63 which are sized and configured to receive complementary projections 65, 67 on the base 45 of the insert 43. In this embodiment, the projections 65, 67 and associated holes 61, 63 have a snap-fit relationship facilitating removable attachment of the insert 43 to the jaw 16. It is appreciated that the holes and projections may vary in width and height for each insert. In this embodiment, the insert 43 is mounted by moving the base 45 downwardly onto the jaw 16 generally along arrow 68. FIG. 5(a) illustrates a perspective view of a clamp jaw 16 having a plurality of holes 61, 63(a)-63(f); FIG. 5(b) illustrates a perspective view of a an insert 43 having a plurality of projections 65, 67(a)-67(f) having a snap-fit relationship with the clamp jaw 16 of FIG. 5(a); FIG. 5(c) illustrates a perspective view of the clamp jaw 16 and the insert 43 in a snap-fit relationship; and FIG. 5(d) illustrates a perspective view of a long and narrow insert 43b having a tissue-contacting surface in accordance with another embodiment of the invention.

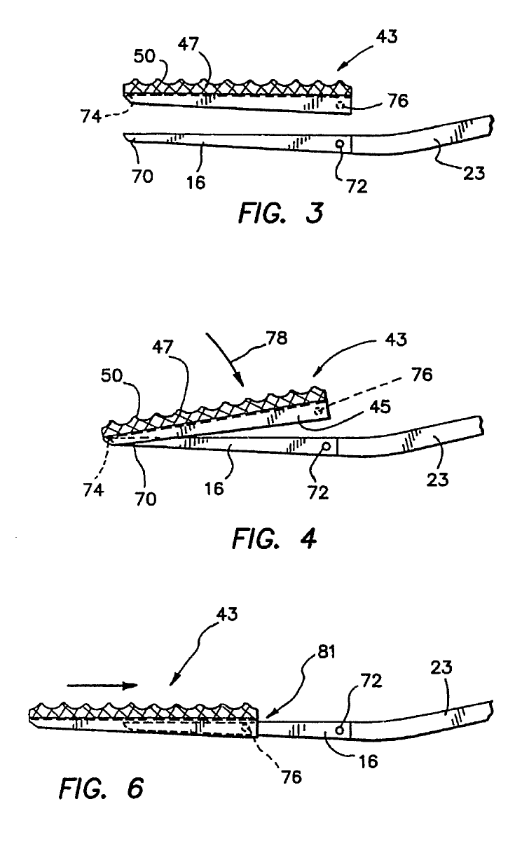

Referring to FIGS. 3 and 4, there are shown side-elevation views of a clamp jaw and system for attachment of a further embodiment of a disposable insert of the invention. In particular, as shown in FIG. 4, a lip 70 is provided at the distal end of jaw 16 and a recess 72 provided along the side surface. The associated insert 43 has a base 45 which is configured with an undercut 74 sized to receive the lip 70, and at least one projection 76 which combines with the recess 72 in a snap-fit relationship. In this case, the insert 43 is mounted by engaging the undercut 74 with the distal lip 70 and then tilting the insert 43 generally in the direction of arrow 78 until the projection 76 snaps into the recess 72.

In still a further embodiment as illustrated in FIG. 6, the insert 43 is provided with a tubular construction including a central channel 81 which is sized and configured to receive the tapered jaw 16. In this embodiment, the tubular insert 43 is mounted by inserting the jaw 16 into the channel 81 and moving the insert 43 relative to the jaw 16 until the projection 76 registers with the recess 72.

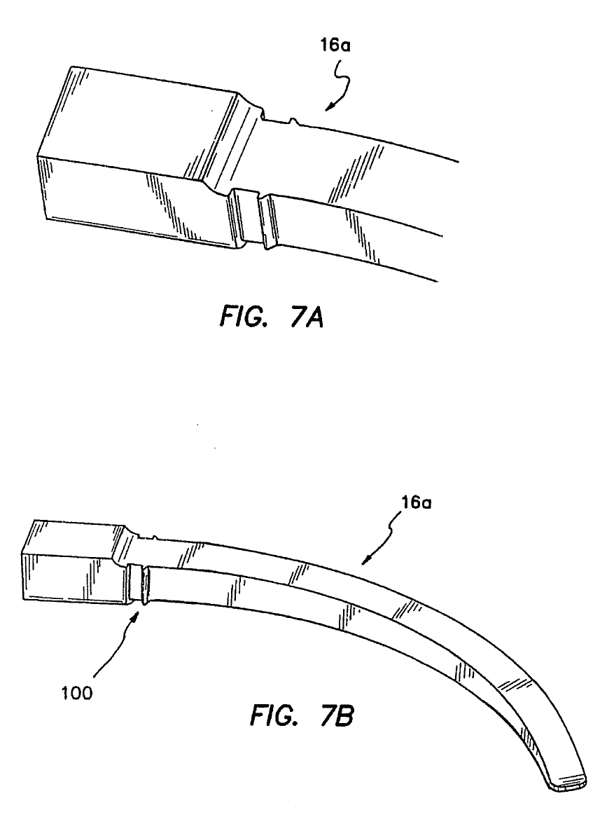

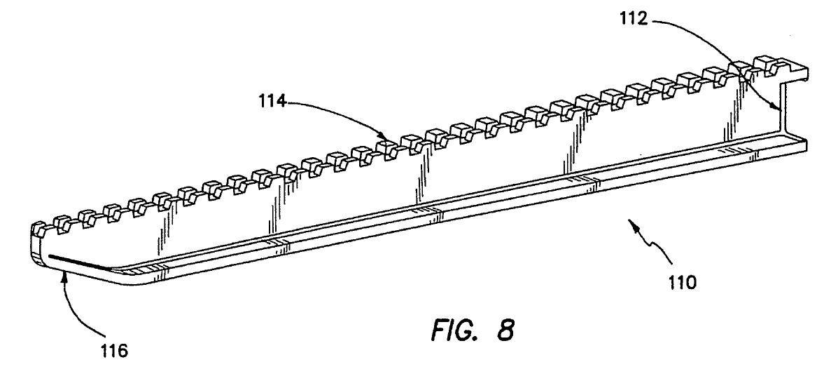

In another aspect of the invention as illustrated in FIG. 7(a), there is shown a jaw 16a having a three-sided barb 100 to aid retention of a flexible insert. It is appreciated that jaw 16a may be bent or curved depending on the application of clamp 10 as illustrated in FIG. 7(b). Referring to FIG. 8, there is shown a section view of an insert 110 having a rectangular molded raised ring 112 to aid the retention of insert 110 over the three-sided barb 100 of jaw 16a. The insert 110 may be easily removed by pulling on the insert in a distal direction to overcome the friction fit of the clamp barb. Together, the jaw 16a having multiple-sided barb 100 and the insert 110 form an enhanced attachment system of the invention providing improved clamping and pulling of small organs, vessels and tissue without the possibility of the insert slipping, twisting or sliding off the clamp jaws in any direction. Other features of the present invention include its low profile and atraumatic clamping capability.

Referring back to FIG. 8, the disposable injection molded insert 110 of the invention may have a thicker wall and a traction pattern 114 at the top on the clamping surface to prevent rollover and to provide atraumatic clamping and traction. The insert 110 may further include a soft tip 116 covering the distal end of the clamp tip or jaw 16a to minimize trauma to internal organs and vessels. The insert 110 provides a snug fit to the clamp tips or jaws to allow for clamping and pulling of small organs, vessels and tissue without the possibility of the insert slipping, twisting or sliding off the clamp jaws in any direction. The insert 110 may be molded in a straight or any configuration and is formed of materials that allow it to flex so it may be used on multiple shaped clamp tips or jaws.

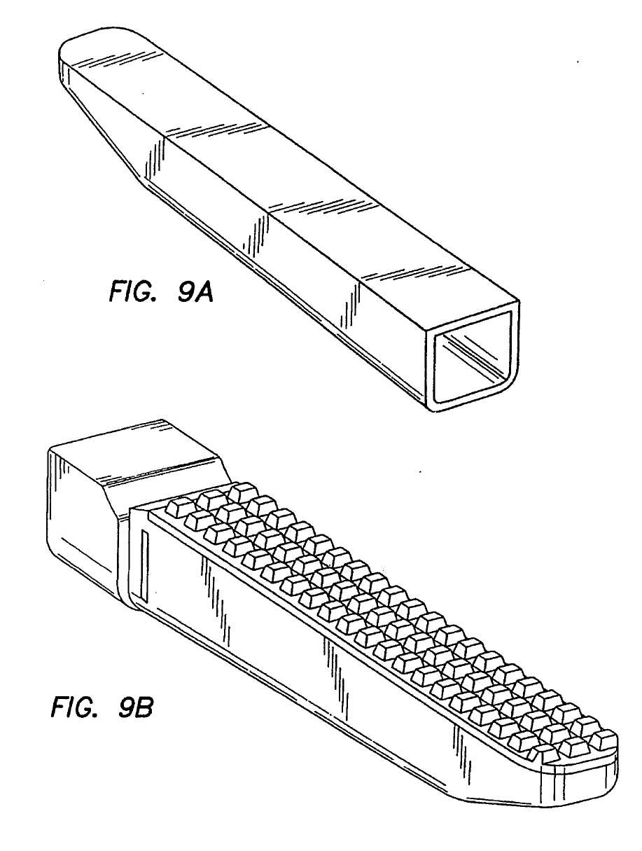

Referring to FIGS. 9(a) and 9(b), there are shown side views or profile of insert 110 and clamp tip or jaw 116a, both of which are tapered to allow for ease of installation and removal. A retention barb 100 may be machined at the proximal end of the clamp tip or jaw 116a to insure adequate retention of the insert 110 during use. The proximal end of the insert 110 has a rectangular molded raised ring 112 to also aid the retention of the insert 110. The inserts 110 may be provided individually sterilized and the clamps may be autoclaved prior to each procedure insuring a sterile environment.

It will be understood that many modifications can be made to the various disclosed embodiments without departing from the spirit and scope of the invention. For example, various sizes of the surgical device are contemplated as well as various types of constructions and materials. It will also be apparent that many modifications can be made to the configuration of parts as well as their interaction. For these reasons, the above description should not be construed as limiting the invention, but should be interpreted as merely exemplary of preferred embodiments. Those skilled in the art will envision other modifications within the scope and spirit of the present invention as defined by the following claims.

1. A surgical instrument for at least partially occluding a body conduit, comprising:
a first jaw having a proximal end and a distal end;
a second jaw opposing the first jaw having a proximal end and a distal end and moveable relative to the first jaw to at least partially occlude the conduit between the first jaw and the second jaw; and
at least one insert having a length, the insert being mounted by the first jaw with the length of the insert extending along the first jaw,
wherein the first jaw further comprises a retention barb to provide a friction fit with the insert.

2. The surgical instrument of claim 1, wherein the insert is formed by injection molding and is disposable.

3. The surgical instrument of claim 1, wherein the retention barb is a three-sided barb.

4. The surgical instrument of claim 1, wherein the retention barb is a multiple-sided barb.

5. The surgical instrument of claim 1, wherein the insert is flexible.

6. The surgical instrument of claim 1, wherein the retention barb is formed at the proximal end of the first jaw.

7. The surgical instrument of claim 1, wherein the first jaw is bent or curved.

8. The surgical instrument of claim 1, wherein the insert comprises an outer wall and an inner wall for fitting over the first jaw, and the inner wall further comprises a molded raised ring to aid the retention of the insert over the barb of the first jaw.

9. The surgical instrument of claim 8, wherein the molded raised ring is formed at the proximal end of the insert.

10. The surgical instrument of claim 8, wherein the molded raised ring is shaped like a rectangle.

11. The surgical instrument of claim 8, wherein the molded raised ring is removed by pulling the insert in a distal direction to overcome the friction fit of the retention barb.

12. The surgical instrument of claim 1, wherein the insert is formed to have a thicker wall and a traction pattern on a clamping surface.

13. The surgical instrument of claim 12, wherein the thicker wall prevents rollover.

14. The surgical instrument of claim 12, wherein the traction pattern provides atraumatic clamping.

15. The surgical instrument of claim 1, wherein the insert further includes a soft tip covering the distal end of the first jaw to minimize trauma during surgery.

16. The surgical instrument of claim 1, wherein the distal ends of the first jaw and the insert are tapered to allow ease of installation and removal.

17. A surgical clamp for at least partially occluding a body conduit, comprising:
a first jaw having a top surface and a bottom surface;
a second jaw opposing the first jaw having a top surface and a bottom surface and moveable relative to the first jaw to at least partially occlude the body conduit; and
at least one insert having a top surface and a bottom surface, the insert being attached to the first jaw with the bottom surface of the insert being removably attached to the top surface of the first jaw,
wherein the bottom surface of the insert comprises at least one or more projections and the top surface of the first jaw comprises at least one or more holes sized and configured to receive said one or more projections of the insert.

18. The surgical clamp of claim 17, wherein the one or more projections of the insert and the one or more holes of the first jaw provide a snap-fit relationship facilitating removable attachment of the insert to the first jaw.

19. The surgical clamp of claim 17, wherein each of the one or more projections of the insert may vary in width and height.

20. The surgical clamp of claim 17, wherein the insert is formed by injection molding and is disposable.
